(19) United States
(12) Patent Application Publication
(10) Pub. No.: US 2018/0049762 A1
(43) Pub. Date: Feb. 22, 2018

(54) ULTRASONIC TRANSDUCER ARRAY FOR SONOTHROMBOLYSIS TREATMENT AND MONITORING

(71) Applicant: KONINKLIJKE PHILIPS N.V., EINDHOVEN (NL)

(72) Inventors: Ralf SEIP, Carmel, NY (US); William Tao SHI, Wakefield, MA (US); Jeffry Earl POWERS, Bainbridge Island, NY (US)

(21) Appl. No.: 15/562,782

(22) PCT Filed: Mar. 29, 2016
(86) PCT No.: PCT/IB2016/051758

§ 371 Date: Sep. 28, 2017

Related U.S. Application Data
(60) Provisional application No. 62/140,018, filed on Mar. 30, 2015.

(51) Int. Cl. A61B 17/225; A61N 7/02; A61B 8/08; A61B 8/00; A61B 90/14

(52) U.S. Cl. CPC A61B 17/2258; A61N 7/02; A61B 8/0891; A61N 2007/0039; A61B 8/0875; A61B 8/488; A61B 90/14; A61B 8/4494

(57) ABSTRACT

An ultrasonic diagnostic imaging system with a two dimensional array transducer performs microbubble-mediated therapy such as sonothrombolysis. The array is formed by dicing into rectilinear elements with the corner elements absent to provide a generally rounded shape that accommodates the temporal windows of the head for cranial therapeutic energy delivery. In several described implementations additional transducer elements are optimized for other specialized functions such as A-line imaging, Doppler flow detection, temporal bone thickness estimation, or cavitation detection. Preferably there are 128 therapeutic elements so that the array probe can be used with standard ultrasound systems having 128-channel beamformers.

This application claims priority to U.S. Prov. Appl. No. 62/140,018, filed on Mar. 30, 2015, which is incorporated by reference herein in its entirety.

This invention relates to medical diagnostic ultrasound systems and, in particular, to ultrasound systems which perform imaging and therapy by sonothrombolysis.

Ischemic stroke is one of the most debilitating disorders known to medicine. The blockage of the flow of blood to the brain can rapidly result in paralysis or death. Attempts to achieve recanalization through thrombolytic drug therapy such as treatment with tissue plasminogen activator (tPA) has been reported to cause symptomatic intracerebral hemorrhage in a number of cases. Advances in the diagnosis and treatment of this crippling affliction are the subject of continuing medical research.

International patent publication WO 2008/017997 (Browning et al.) describes an ultrasound system which provides microbubble-mediated therapy to a thrombus such as one causing ischemic stroke. Microbubbles are infused, delivered in a bolus injection, or developed into the bloodstream and flow to the vicinity of a thrombus. Ultrasound energy is delivered to the microbubbles at the thrombus to disrupt or rupture the microbubbles. This microbubble activity can in many instances aid in dissolving or breaking up the blood clot and return a nourishing flow of blood to the brain and other organs. Such microbubble activity can be used to deliver drugs encapsulated in microbubble shells, and well as microbubble-mediated sonothrombolysis.

The Browning et al. publication shows the ultrasonic energy being delivered for sonothrombolysis from an ultrasound array probe controlled by an ultrasound system. For sonothrombolysis treatments to be clinically safe and effective, the ultrasound array probe delivering the ultrasound energy to the clot target region should meet various requirements. First, the probe must be capable of adequate ultrasound energy delivery at the clot site, sufficient to stimulate sonothrombolytic activity in arteries within the brain. Second, the energy delivery should be directionally controllable, providing the capability to target the tissue surrounding the clot. The energy delivered should be controllable, providing the ability to reach both deep and shallow clots. The array should be sized and shaped to fit an acoustic window of the skull, and preferably have the ability to indicate correct placement on the patient's temporal bone window. Finally, the system should provide the capability to estimate the in-situ pressure for proper ultrasound dose delivery and enhanced treatment safety.

In accordance with the principles of the present invention, a transducer array and ultrasound system are described which provide the ability to perform sonothrombolytic treatment using a standard 128-channel beamformer. The transducer array in the probe is a two dimensional array so that the energy delivery can be controllably directed in three dimensions. The array is generally rounded and shaped to fit the temporal bone window of a patient's head. Exemplary transducer arrays are described which can be powered by a standard system beamformer, capable of delivering sufficient energy to stimulate sonothrombolysis. Implementations are described with imaging transducer elements that are, in combination with the ultrasound systems, optimized for functionality other than therapeutic energy delivery, such as A-line imaging, Doppler detection, skull thickness ranging, or sensitivity to signals characteristic of cavitation.

In the drawings:

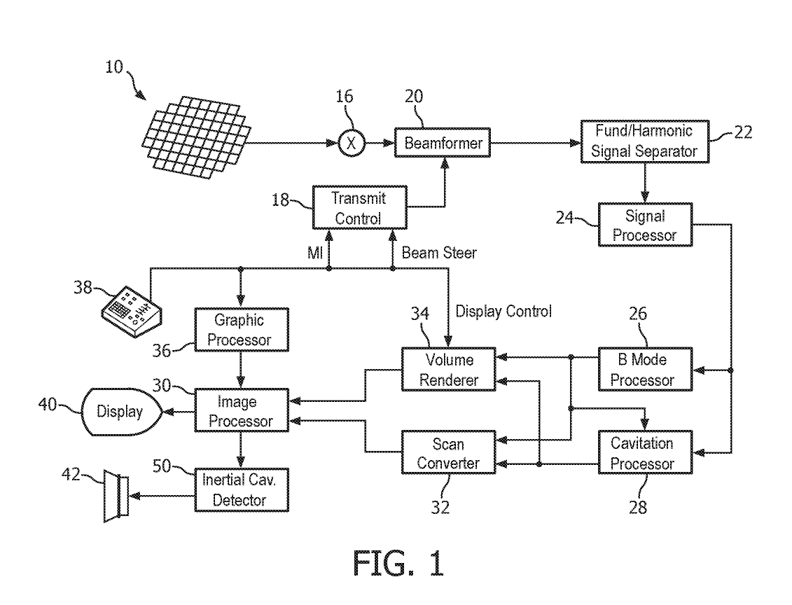

FIG. 1 illustrates in block diagram form an ultrasonic diagnostic imaging and therapy system constructed in accordance with the principles of the present invention.

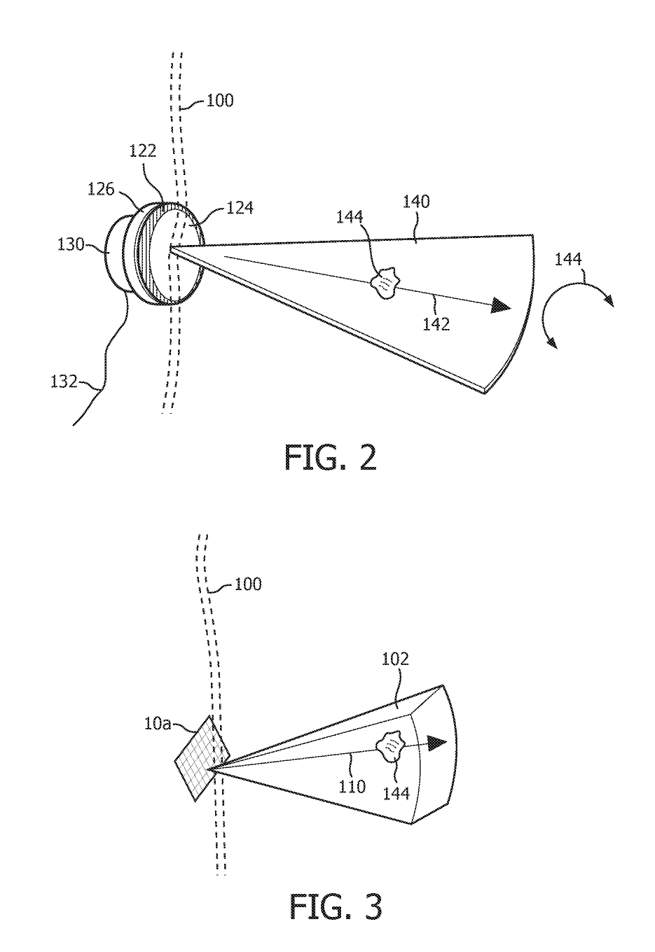

FIG. 2 illustrate the delivery of sonothrombolysis therapy in a two dimensional (2D) imaging plane

FIG. 3 illustrates the delivery of sonothrombolysis therapy in a three dimensional image volume.

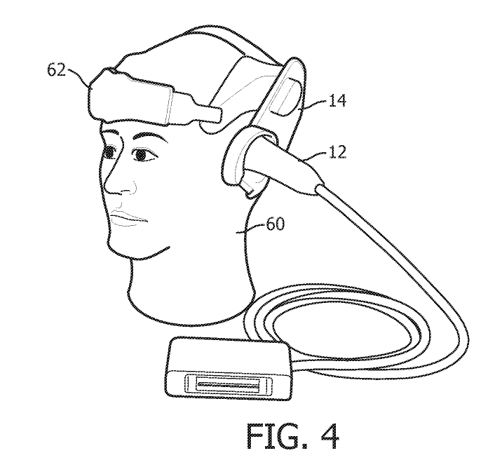

FIG. 4 illustrates a probe and headset for sonothrombolysis therapy modeled on the head of a mannequin.

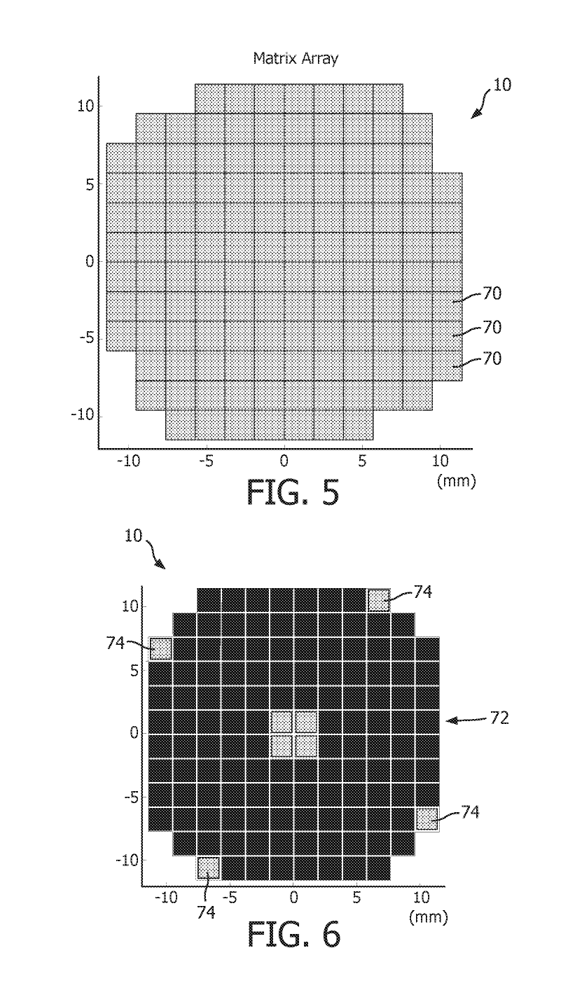

FIG. 5 illustrates a two dimensional transducer array constructed in accordance with the principles of the present invention.

FIG. 6 illustrates another two dimensional array of the present invention with central receive-only elements.

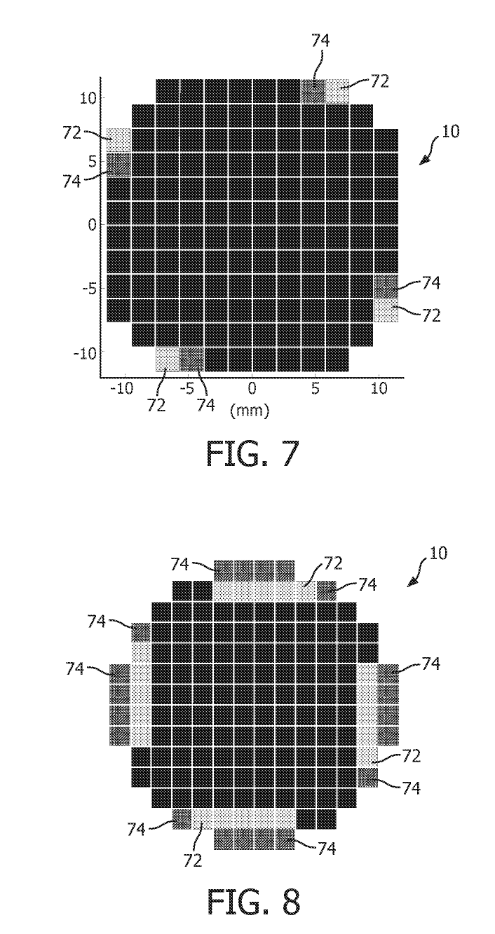

FIG. 7 illustrates another two dimensional array of the present invention with peripheral receive-only elements.

FIG. 8 illustrates another two dimensional array of the present invention with peripheral receive-only elements.

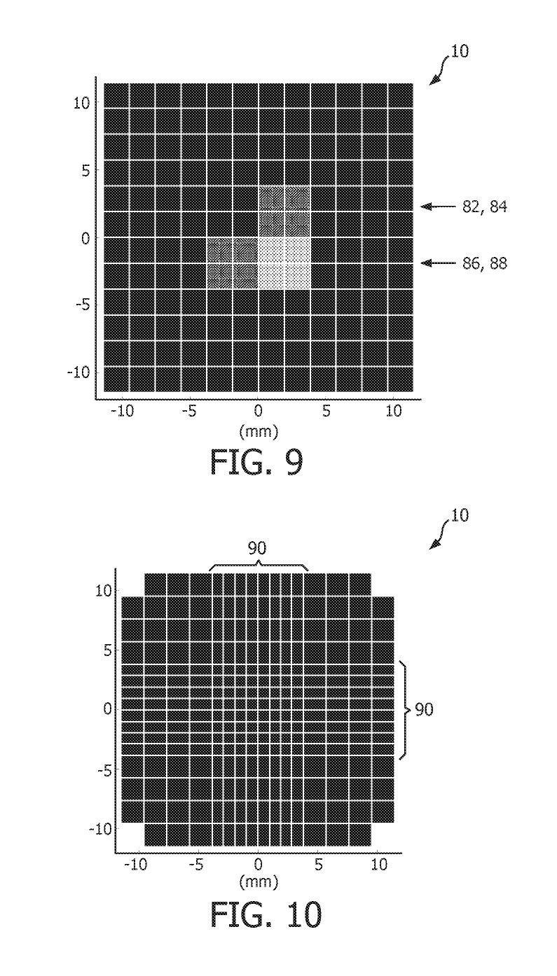

FIG. 9 illustrates a two dimensional array of the present invention with four dedicated central elements.

FIG. 10 illustrates another two dimensional array of the present invention with finer pitch imaging elements.

In some aspects, the present invention includes an ultrasonic therapy system comprising instructions thereon that when executed cause the system to transmit therapeutic ultrasound energy from a two dimensional array of therapy transducer elements toward an occlusion in a cranial vascular system, and transmit other than therapeutic ultrasound energy from imaging transducer elements positioned with the two dimensional array of therapy transducer elements. The two dimensional array can include rectilinearly diced transducer elements arranged in a pattern with corner elements missing to provide a generally rounded array shape.

In certain aspects, a number of therapy transducer elements in the two dimensional array is 128, and the ultrasonic therapy system further includes a 128-channel beamformer. The imaging transducer elements can be centrally positioned in the two dimensional array of therapy ultrasound elements. In some aspects, the imaging transducer elements are peripherally positioned around the two dimensional array of therapy transducer elements. The number of imaging elements can range, and can be generally less than the number of therapy transducer elements. For example, a number of the imaging transducer elements is four. In some aspects, twenty imaging transducer elements arranged in groups of five elements, each group being located on a side of the two dimensional array of therapy transducer elements In certain aspects, the imaging transducer elements (e.g., four elements) can be coupled together for operation in parallel as a transducer patch. In certain aspects, the imaging transducer elements can be peripherally positioned around the two dimensional array of therapy transducer elements, and, alternatively, the imaging transducer elements are coupled together for operation in parallel.

In certain aspects, the system can include instructions that when executed cause the imaging transducer elements to transmit ultrasound at a higher frequency than the therapy transducer elements, and/or the imaging transducer elements can be structurally configured to operate at a higher frequency than the therapy transducer elements. For example, the imaging transducer elements can include a smaller height than the therapy transducer elements. In some aspects, the imaging transducer elements can also include a heavier backing for wider bandwidth and/or a different acoustic matching layer for different energy coupling into a body. As described further herein, the imaging transducer elements and the ultrasound system can be configured for one of A-line imaging, Doppler detection, or skull thickness ranging. The imaging transducer elements can also have a bandwidth sensitive to sub- or ultraharmonic frequencies characteristic of cavitation. In certain aspects, the ultrasonic therapy system can include a cavitation detector, responsive to signals produced by the imaging transducer elements, and amplifier electronics that are coupled to the two dimensional array and configured to control the ultrasonic energy produced by the therapy transducer elements.

Referring to FIG. 1, an ultrasound system constructed in accordance with the principles of the present invention is shown in block diagram form. A two dimensional transducer array 10 is provided for transmitting ultrasonic waves for therapy and other uses as described below and receiving echo information. In this invention the array is a two dimensional array of transducer elements that in combination with an ultrasound system are capable of steering ultrasound waves having therapeutic effect in three dimensions and providing 3D image and other information. In this example the array is located in an ultrasound probe which mounts on a headset that locates the array in acoustic contact with the temple on the side of the head for transcranial delivery of sonothrombolysis. The elements of the array are coupled to a transmit/receive (T/R) switch 16 which switches between transmission and reception and protects the system beamformer 20 from high energy transmit signals. The transmission of ultrasonic pulses from the transducer array 10 is directed by the transmit controller 18 coupled to the beamformer 20, which receives input from the user's operation of the user interface or control panel 38.

The echo signals received by elements of the array 10 are coupled to the system beamformer 20 where the signals are combined into coherent beamformed signals. For example, the system beamformer 20 in this example has 128 channels, each of which drives an element of the array to transmit energy for therapy or imaging, and receives echo signals from one of the transducer elements. In this way the array is controlled to transmit steered beams of energy and to steer and focus received beams of echo signals.

The beamformed receive signals are coupled to a fundamental/harmonic signal separator 22. The separator 22 acts to separate linear and nonlinear signals so as to enable the identification of the strongly nonlinear echo signals returned from microbubbles or tissue. The separator 22 may operate in a variety of ways such as by bandpass filtering the received signals in fundamental frequency and harmonic frequency bands (including super-,sub-, and/or ultra-harmonic signal bands), or by a process for fundamental frequency cancellation such as pulse inversion or amplitude modulated harmonic separation. Other pulse sequences with various amplitudes and pulse lengths may also be used for both linear signal suppression and nonlinear signal enhancement. A suitable fundamental/harmonic signal separator is shown and described in international patent publication WO 2005/074805 (Bruce et al.) The separated signals are coupled to a signal processor 24 where they may undergo additional enhancement such as speckle removal, signal compounding, and noise elimination.

The processed signals are coupled to a B mode processor 26 and a cavitation processor 28. The B mode processor 26 employs amplitude detection for the imaging of structures in the body such as muscle, tissue, and blood cells. B mode images of structure of the body may be formed in either the harmonic mode or the fundamental mode. Tissues in the body and microbubbles both return both types of signals and the stronger harmonic returns of microbubbles enable microbubbles to be clearly segmented in an image in most applications. A cavitation processor 28 detects signal characteristics of cavitation and produces cavitation image and alert signals as described below. The system may also include a Doppler processor which processes temporally distinct signals from tissue and blood flow for the detection of motion of substances in the image field including red blood cells and microbubbles. The anatomic and cavitation signals produced by these processors are coupled to a scan converter 32 and a volume renderer 34, which produce image data of tissue structure, flow, cavitation, or a combined image of several of these characteristics. The scan converter converts echo signals with polar coordinates into image signals of the desired image format such as a sector image in Cartesian coordinates. The volume renderer 34 converts a 3D data set into a projected 3D image as viewed from a given reference point as described in U.S. Pat. No. 6,530,885 (Entrekin et al.) As described therein, when the reference point of the rendering is changed the 3D image can appear to rotate in what is known as kinetic parallax. This image manipulation is controlled by the user as indicated by the Display Control line between the user interface 38 and the volume renderer 34. Also described is the representation of a 3D volume by planar images of different image planes, a technique known as multiplanar reformatting. The volume renderer 34 can operate on image data in either rectilinear or polar coordinates as described in U.S. Pat. No. 6,723,050 (Dow et al.) The 2D or 3D images are coupled from the scan converter and volume renderer to an image processor 30 for further enhancement, buffering and temporary storage for display on an image display 40.

A graphics processor 36 is also coupled to the image processor 30 which generates graphic overlays for displaying with the ultrasound images. These graphic overlays can contain standard identifying information such as patient name, date and time of the image, imaging parameters, and the like, and can also produce a graphic overlay of a beam vector steered by the user as described below. For this purpose the graphics processor received input from the user interface 38. In an embodiment of the present invention the graphics processor can be used to overlay a cavitation image over a corresponding anatomical B mode image. The user interface is also coupled to the transmit controller 18 to control the generation of ultrasound signals from the transducer array 10 and hence the images produced by and therapy applied by the transducer array. The transmit parameters controlled in response to user adjustment include the MI (Mechanical Index) which controls the peak intensity of the transmitted waves, which is related to cavitational effects of the ultrasound, and steering of the transmitted beams for image positioning and/or positioning (steering) of a therapy beam as discussed below.

FIG. 2 illustrates the conduct of sonothrombolysis in two dimensions with a one dimensional transducer array. In this example the transducer array 122 is a one dimensional array which performed 2D imaging. This transducer array, like the other arrays described herein, is covered with a lens 124 which electrically insulates the patient from the transducer array and in the case of a one dimensional array may also provide focusing in the elevation (out-of-plane) dimension. The lens is pressed against the skinline 100 for acoustic coupling to the patient. The transducer array 122 is backed with air or acoustic damping material 126 which attenuates acoustic waves emanating from the back of the array to prevent their reflection back into the transducer elements. Behind this transducer stack is a device 130 for rotating the image plane 140 of the array. The device 130 may be a simple knob or tab which may be grasped by the clinician to manually rotate the circular array transducer in its rotatable transducer mount (not shown). The device 130 may also be a motor which is energized through a conductor 132 to mechanically rotate the transducer as discussed in U.S. Pat. No. 5,181,514 (Solomon et al.) Rotating the one dimensional array transducer 122 as indicated by arrow 144 will cause its image plane 140 to pivot around its central axis, enabling the repositioning of the image plane for full examination of the vasculature in front of the transducer array. As discussed in the '514 patent, the planes acquired during at least a 180° rotation of the array will occupy a conical volume in front of the transducer array, which may be rendered into a 3D image of that volumetric region. Other planes outside this volumetric region may be imaged by repositioning, rocking or tilting the transducer array in its headset in relation to the skull 100. If a stenosis, a blood clot, is found in the image of the plane being imaged, the therapeutic beam vector graphic 142 can be steered by the clinician to aim and focus the beam at the stenosis 144 and therapeutic pulses applied to disrupt the microbubbles at the site of the stenosis.

FIG. 3 illustrates a 3D imaging/therapy implementation of the present invention which uses a 2D matrix array transducer 10a. In this illustration the transducer array 10a is held against the skinline 100 of the patient with the volume 102 being imaged projected into the body. The user will see a 3D image of the volume 102 on the display of the ultrasound system in either a multiplanar or volume rendered 3D projection. The user can manipulate the kinetic parallax control to observe the volume rendered 3D image from different orientations. The user can adjust the relative opacity of the tissue and flow components of the 3D image to better visualize the vascular structure inside the brain tissue as described in U.S. Pat. No. 5,720,291 (Schwartz) or can turn off the B mode (tissue) portion of the display entirely and just visualize the flow of the vascular structure inside the 3D image volume 102.

When the site of the treatment such as a thrombus 144 is being imaged in the volume 102, a microbubble contrast agent is introduced into the patient's bloodstream. In a short time the microbubbles in the bloodstream will flow to the vasculature of the treatment site and appear in the 3D image. Therapy can then be applied by agitating or breaking microbubbles at the site of the stenosis in an effort to dissolve the blood clot. The clinician activates the “therapy” mode, and a therapy graphic 110 appears in the image field 102, depicting the vector path of a therapeutic ultrasound beam with a graphic thereon which may be set to the depth of the thrombus. The therapeutic ultrasound beam is manipulated by a control on the user interface 38 until the vector graphic 110 is focused at the site of the blockage. The energy produced for the therapeutic beam can be within the energy limits of diagnostic ultrasound or in excess of the ultrasound levels permitted for diagnostic ultrasound. The energy of the resulting microbubble ruptures will strongly agitate a blood clot, tending to lyse the clot and dissolve it in the bloodstream. In many instances insonification of the microbubbles at diagnostic energy levels will be sufficient to dissolve the clot. Rather than breaking in a single event, the microbubbles may be vibrated and oscillated, and the energy from such extended oscillation prior to dissolution of the microbubbles can be sufficient to lyse the clot.

FIG. 4 illustrates a headset 62 for a sonothrombolysis array probe 12 of the present invention mounted on the head 60 of a mannequin. The sides of the head of most patients advantageously provide suitable acoustic windows for transcranial ultrasound at the temporal bones around and in front of the ears on either side of the head. In order to transmit and receive echoes through these acoustic windows the transducer arrays must be in good acoustic contact at these locations which may be done by holding the transducer arrays against the head with the headset 62. An implementation of the present invention may have a snap-on deformable acoustic standoff which allows the transducer array to be manipulated by its conformal contact surface and aimed at the arteries within the brain while maintaining acoustic contact against the temporal window. An array 10 of the present invention is integrated into the probe housing 12 which allows it to address the requirement of stable positioning and tight coupling to the patient's temporal bone. The illustrated probe housing is curved by bending the probe handle by 90°, which makes the probe more stable when attached to the headset 62. The acoustic coupling objective is facilitated by integrating a mating spherical surface into the probe handle, which allows it to pivot in the headset 62 until it is strongly and tightly coupled to the temporal window of the patient.

Existing transcranial probes are designed for imaging and flow diagnostic purposes. As such, these probes tend to be higher-frequency (center frequency generally in the range of 1.6 to 2.5 MHz) probes, utilizing wide bandwidth piezoelectric transducer elements meeting the λ/2 size requirement. These probes generate reasonable ultrasound images of the brain and its vasculature, but at a cost of penetration depth, efficiency, and output power. Furthermore, most of these probes are also not specifically designed to be used transcranially, thus not taking advantage of the full (either mostly circular or ellipsoidal) aperture (typically 2-2.5 cm) that the temporal bone window provides, resulting in further reduced output power due to a smaller probe aperture. In accordance with the principles of the present invention, the array transducer 10 is formed as a generally rounded array 10 of 128 therapy elements 70 as shown in FIG. 5. The generally rounded shape fits well with the rounded shape of the temporal bone acoustic window on the side of the head. In a constructed implementation the individual elements are relatively sizeable, exhibiting a pitch of approximately 2 mm. Simulations and measurements indicate that the array is able to reach clots located at depths exceeding 60-65 mm, thus able to meet the clot targeting objective listed above. This allows the matrix array to reach up to 97.7% of middle-cerebral artery clots. Because the individual array elements are large, their electrical impedance is lower than elements of conventional arrays, facilitating electrical impedance matching. The use of large, highly resonant elements (with air or other light backing material for efficient power transfer) also allows the array to generate significant output power/pressure for prolonged periods of time, e.g., several tens of milliseconds, found to be optimal for clot dissolution. The transmit efficiency is also required to achieve in-situ pressures in the brain of approximately 300-500 kPa, while still being able to overcome the significant attenuation from the temporal bone and intervening brain tissue, which on average can reduce the incident pressure by a factor of 3-4. The illustrated arrangements of elements, as well as their size, enables off-axis steering at up to +/−27° to target clots that are not located directly in front of the array aperture, and to target the tissue surrounding the clot, another one of the objectives listed above. The individual elements themselves are arranged in rows and columns to facilitate their fabrication by a dicing process, but are absent from the corners of the array to provide a generally rounded shape to the array.

A basic array 10 of the present invention is shown in FIG. 5. The array comprises 128 elements 70, meaning it can be operated by a standard 128-channel beamformer of the typical ultrasound system. The 128 elements are operated together to steer and focus therapeutic energy at microbubbles and blood clots in the brain. At each corner of the array four elements are missing from the otherwise rectilinear shape to give the array its generally rounded form that fits the temporal bone acoustic window. FIG. 6 illustrates a modified form of the standard array in which the four central elements 72 are dedicated to a function separate from the 128-element therapy array. The four center elements 72 can be coupled together electrically to form a single, larger element “patch”. This has the advantage of providing higher sensitivity, only requiring a single channel from an ultrasound system either for pulse-echo operation as may be used for skull bone ranging purposes, for operating in receive-mode only as may be required in a passive cavitation detection system, or for operating in a pulsed Doppler mode as may be required for blood flow (or absence of blood flow) detection. Such a small element patch has the additional advantage of not being very directive. Thus, such a patch is sensitive to receiving ultrasound signals originating from a large volume in front of the sensor, which is beneficial for cavitation detection. The four central elements 72 thus act as a separate single-element transducer. The function of the central elements can be A-line imaging/detection/ranging, or passive cavitation detection, for instance. These elements can thus be optimized to operate at a higher frequency more suitable for trans-cranial imaging (e.g., 1.6-2.5 MHz), or detection of the harmonic of the transmitted signal (e.g. 2 MHz) but manufactured during the same manufacturing process as the main therapeutic array. Simple modifications can be applied to only this subset of elements such as a smaller height, resulting in a higher operating frequency; a heavier backing, resulting in a wider bandwidth; or a different acoustic matching layer, resulting in better energy coupling into the body at their specific frequency of operation. The dedication of the four center elements to another function means that the therapeutic array now has only 124 elements. To fully utilize all of the channels of a standard beamformer, four new elements can be added to the therapy array during the manufacturing process, such as peripheral elements 74.

FIG. 7 illustrates another array configuration, in which the specially-dedicated elements 72 are located around the periphery of the array 10. In this implementation the elements 72 are replaced in the 128-element therapeutic array with four elements 74, maintaining the full count of 128 elements in the therapeutic array.

FIG. 8 shows another implementation of an array of the present invention in which five elements 72 on each side of the array 10 are electrically coupled together and used for a different function such as ranging or cavitation detection. The dedication of these twenty elements reduces the element count of the therapeutic array to 108, a number which is increased back to 128 by the addition of five therapy elements 74 on each side of the array, four as a new outer row and one added to the former outer row.

In the manufacture of a transducer array of the present invention, a 2D ultrasound array is fabricated in the usual manner (e.g., lapping, dicing, etc.) with the characteristics of each of the elements fine-tuned for the sonothrombolysis therapeutic application, e.g., 1 MHz, 2-6 cm depth focusing, +/−27o off-axis steering capability, narrow bandwidth, high efficiency, high output power, circular aperture. A subset of the elements of the array is set aside and fine-tuned so their electrical and acoustic characteristics match a special application, e.g., 1.6-2.0 MHz, wide bandwidth, high sensitivity for A-line imaging, Doppler detection, or skull thickness ranging. Alternatively, the electrical and acoustic characteristics of the subset of elements are fine-tuned to be sensitive to sub- or ultraharmonic frequencies of the main therapeutic frequency to enable better detection of these frequencies for implementing a passive cavitation detection functionality. The specialized elements are combined electrically or acoustically to form an element patch which, while narrowing their directivity, increases their sensitivity to the desired signals.

In use, the therapeutic elements are powered to deliver the sonothrombolysis therapy, focusing the array on the clot target and surrounding tissue. The subset of specialized elements is used to

a. Gauge the quality of the temporal bone window by examining the amplitude of the echo reflected from the contralateral side of the skull. A larger amplitude implies a thinner temporal bone window, and/or a better position for the entire array on the temporal bone window.

b. Determine the flow and/or absence of flow of the middle-cerebral artery by operating the patch in Doppler mode, to help in targeting the sonothrombolysis beam to the occlusion.

c. Determine the thickness of the temporal bone window directly by use of a high-frequency patch, e.g., 10-20 MHz. This information is used to modulate the output power of the sonothrombolysis therapeutic array: a thinner temporal bone window would require a lower sonothrombolysis output pressure in order to achieve the same in-situ pressure as compared to a thicker temporal bone window. Or,

d. Determine the in-situ pressure by listening to the signal emanating from the microbubbles while being subjected to the sonothrombolysis treatment frequency, via detection/classification of the spectrum of the returning signal by the cavitation processor 28. If the signature for inertial cavitation is detected, for example, and stable cavitation is desired, the inertial cavitation detector 50 produces an alarm by a speaker 42. The user responds to this information by reducing the ultrasound output power (MI) being generated by the sonothrombolysis array. If cavitation is not detected at all, for example, by no indication of cavitation coloring of the site of the occlusion in the image by the cavitation processor 28, then the output power of the sonothrombolysis array is increased until cavitation is detected. This output power scaling can also be accomplished automatically without user intervention via an output power control loop, for example. The treatment is continued at this setting. Such usage allows the system to compensate for the attenuation generated by different temporal bone windows and any varying attenuation due to different acoustic properties of brain tissue.

The transducer array of FIG. 9 illustrates an arrangement with several sub-patches 82-88, each fine-tuned to a specific frequency for best operation of its specialized function. For instance, patch 82 operates at 1.6-2.0 MHz for ranging and temporal bone quality determination; a second patch 84 operates at 10-20 MHz for direct temporal bone thickness estimation; a third patch 86 operates at 3 MHz for harmonic detection; and a fourth patch 88 operates at 5 MHz for Doppler flow detection. Each of the sub-patches 82-88 can be connected to and driven by its own imaging/detection subsystem, or it can be connected to an individual ultrasound system front-end channel, as needed. In this example, the sonothrombolysis therapeutic array 10 composed of the surrounding elements is still made up of 128 elements, thus continuing to utilize the transmitter and amplifier electronics of the ultrasound system in its most complete and efficient way. The central location of the imaging/detection subpatches 82-88 allows them to be pointed generally in the same direction, thus covering mostly the same volume/region of the brain.

The concepts of the present invention can be extended to patches consisting of more or less than four elements and overall matrix array geometries with more than 128 elements. Geometries such as that shown in FIG. 10, where even the element size of the patch elements is different than those of the therapeutic array, can be implemented with current ceramic dicing technology using linear dicing cuts. In the example of FIG. 10, the smaller rectangular elements of the array outlined at 90 are interconnected electrically to re-form them into larger square elements, matching the size of those that make up the rest of the geometry of the therapeutic array. Thus the full array can be used for sonothrombolysis treatment. The smaller central elements of the patch can be wired together to either act as a single-element transducer patch (i.e., all in parallel), or separately, so that each element is connected to its own pulser/receiver channel or driving electronics, to implement a two dimensional, small pitch, matrix array for two or three dimensional imaging. This would further optimize the central sub-array for a specific application (imaging, ranging, color Doppler, flow detection, etc.) by adding a capable focusing and/or beam steering functionality to the device.

It should be noted that the various embodiments described above and illustrated by drawings may be implemented in hardware, software or a combination thereof. The various embodiments and/or components, for example, the modules, or components and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasonic therapy system comprising instructions thereon that when executed cause the system to:
transmit therapeutic ultrasound energy from therapy transducer elements toward an occlusion in a cranial vascular system, wherein the therapy transducer elements are included in a two dimensional array, the two dimensional array comprising rectilinearly diced transducer elements arranged in a pattern with corner elements missing to provide a generally rounded array shape; and
transmit other than therapeutic ultrasound energy from imaging transducer elements, where in the imaging transducer elements are included in the two dimensional array,
wherein transmission and reception of ultrasound energy by the therapy transducer elements and imagine transducer elements are coupled to a single beamformer.

2. The transducer array of claim 1, wherein a number of therapy transducer elements in the two dimensional array is 128 and wherein the beamformer comprises a 128-channel beamformer.

3. The transducer array of claim 2, wherein the imaging transducer elements are centrally positioned in the two dimensional array of therapy ultrasound elements.

4. The transducer array of claim 3, wherein a number of the imaging transducer elements is four.

5. The transducer array of claim 4, wherein the four imaging transducer elements are coupled together for operation in parallel as a transducer patch.

6. The transducer array of claim 2, wherein the imaging transducer elements are peripherally positioned around the two dimensional array of therapy transducer elements.

7. The transducer array of claim 6, wherein the imaging transducer elements are coupled together for operation in parallel.

8. The transducer array of claim 6, wherein a number of imaging transducer elements is four.

9. The transducer array of claim 6, comprising twenty imaging transducer elements arranged in groups of five elements, each group being located on a side of the two dimensional array of therapy transducer elements.

10. The transducer array of claim 1, wherein the system comprises instructions that when executed cause the imaging transducer elements to transmit ultrasound at a higher frequency than the therapy transducer elements.

11. The transducer array of claim 10, wherein the imaging transducer elements comprise a smaller height than the therapy transducer elements.

12. The transducer array of claim 1, wherein the imaging transducer elements comprise one or more of a heavier backing for wider bandwidth or a different acoustic matching layer for different energy coupling into a body.

13. The transducer array of claim 1, wherein the imaging transducer elements are configured for one of A-line imaging, Doppler detection, or skull thickness ranging.

14. The transducer array of claim 1, wherein the imaging transducer elements comprise a bandwidth sensitive to sub- or ultraharmonic frequencies characteristic of cavitation.

15. The ultrasonic therapy system of claim 14, wherein the ultrasonic therapy system further comprises:
a cavitation detector, responsive to signals produced by the imaging transducer elements; and amplifier electronics that are coupled to the two dimensional array and configured to control the ultrasonic energy produced by the therapy transducer elements.
